(19) United States
(12) Patent Application Publication
(10) Pub. No.: US 2005/0118233 A1
(43) Pub. Date: Jun. 2, 2005

(54) Dieting system and method based on controlled carbohydrate intake

(76) Inventors: Baruch Segal, Moshav Beit Nehemia (IL); Yosef Shmuel Daniel, Yerucham (IL)

Correspondence Address: DR. MARK FRIEDMAN LTD.;C/o Bill Polkinghorn, Discovery Dispatch, 9003 Florin Way, Upper Marlboro, MD, 20772, US

(21) Appl. No.: 10/725,005

(22) Filed: Dec. 2, 2003

(51) Int. Cl. A47G 19/00; A61K 47/00; A23G 3/00

(52) U.S. Cl. 424/439; 426/658; 220/575

(57) ABSTRACT

A system for reducing human body weight to a permanent optimal weight level comprises a portable kit that includes a plurality of foods with a known total carbohydrate content altanged in an asymmetric spatial arrangement, and spoiling prevention means for keeping the foods from spoiling. The asymmetric arrangement preferably includes a plurality of containers holding the foods and organized such that similar carbohydrate content foods are stored by groups, from a group of largest number of containers with carbohydrate free foods to preferably a single container with a carbohydrate rich food. Optionally, the groups are marked by different- colors to provide additional identification of the carbohydrate content.

### FIELD AND BACKGROUND OF THE INVENTION

The present invention relates to dieting systems and methods, and, more specifically, to systems and methods that restrict daily carbohydrate intake by providing food kits with defined carbohydrate content.

The widespread problems of obesity and overweight in humans have attracted the attention of the medical and nutrition communities through the past few decades. The answers to these problems lie both in the realm of medical treatments, through drugs and medical operations, and in that of nutrition, through various diet programs and methods. While drastic medical measures may indeed lead to permanent weight loss, most diet programs and methods fail to produce long lasting results. In particular, programs based on the restriction of the diet to certain foods while eliminating other diet ingredients, or on restricting calories, fail to yield permanent beneficial weight loss. This is particularly true for obese or grossly overweight persons.

Low carbohydrate diets are known. Particularly prominent is the Adkins diet, which is based on the theory that overweight people eat too many carbohydrates. The human body burns both fat and carbohydrates for energy, but carbohydrates are used first. By drastically reducing the carbohydrate intake, and by eating more protein and fat, the human body loses weight naturally because it burns stored body fat more efficiently.

The Adkins diet sets few limits on the amount of food one can eat but instead severely restricts the kinds of food allowed to foods traditionally regarded as “rich”, e.g. meat, eggs, cheese, etc., while forbidding refined sugar, milk, white rice, or white flour. The diet has a mandatory two-week induction period of extreme restriction on the carbohydrate intake to less than 20 grams per day. This induces a state of ketosis, which means the body burns its own fat for fuel. The ketosis state is one major disadvantage of the Adkins diet, having by itself, or in combination with other aspects of this diet, significant neoative health-impacting side effects.

There is thus a widely recognized need for, and it would be highlly advantageous to have, an efficient diet system and method, which does not restrict the type of food ingredients a person can eat, leaving a well-balanced diet, while providing significant permanent weight loss. There is also a need for a low-carbohydrate diet that will not induce ketosis, thus avoiding the kinds of negative side effects present in existing low carbohydrate diets.

### SUMMARY OF THE INVENTION

According to the present invention there is provided a system for reducing human body weight to a permanent optimal weight level, comprising a portable kit that includes a plurality of foods with a known total carbohydrate content, and spoiling prevention means for keeping the foods from spoiling.

According to the present invention there is provided a method for permanently reducing body weight in humans, comprising the steps of determining an optimal daily carbohydrate intake, organizing foods in a portable device according to the optimal carbohydrate intake, and freely consuming the foods from the kit throughout the day.

According to the present invention there is provided a portable dieting apparatus comprising an asymmetrically shaped kit that includes a plurality of food storage units, and a plurality of foods having a known total carbohydrate content and stored in the storage units according to a carbohydrate content order.

According to one feature in the portable dieting apparatus of the present invention, the kit further comprises spoiling prevention means to keep said foods from spoiling.

According to yet another feature in the portable dieting apparatus of the present invention, the kit further comprises color-coding means for marking the carbohydrate content order.

### BRIEF DESCRIPTION OF THE DRAWINGS

The invention is herein described, by way of example only, with reference to the accompanying drawings, wherein:

FIG. 1 is a photograph of a preferred embodiment of a diet kit according to the present invention: a) internal view; b) external view.

FIG. 2 shows in (a) an embodiment of a kit comprising an asymmetrical one-piece enclosure having a plurality of compartments, and in (b) a one-piece enclosure kit with cooling packs inserted in spaces between the compartments.

### DESCRIPTION OF THE PREFERRED EMBODIMENTS

The present invention is of a new type of diet system and method, based on controlled carbohydrate daily intake, which provides a dieting person a full range of foods, yet leads to significant permanent weight loss. In other words, the controlled carbohydrate intake system and method disclosed herein allow to bring a human body weight to a permanent optimal weight level. The invention is based on the premise that most existing diet systems and methods fail because they entail a dieting regime that is difficult to follow over a long period of time. Even with the best intentions and will power, an obese or overweight person who wants to follow known dieting regimes eventually slacks off and gives up, normally because the regime appears to be ineffective after a while, or has requirements that are too draconian or induce unwanted side effects. In contrast, the present invention allows a person to eat the entire normal range of foods, but ensures that the total daily carbohydrate intake is limited, so that over time there is a gradual loss of weight, eventually stabilizing in a permanent, normal weight. Moreover, the present invention leads to permanent weight loss even in persons that do not engage in physical activity. In contrast with other low-carbohydrate diets, and in particular with the Adkins diet, the method of the present invention does not require a state of ketosis at any time, and therefore removes a major source of negative side-effects.

The present invention is based on the concept of a “carbohydrate bank” from which the dieting person may eat any food up to a given total daily carbohydrate content. We will refer to this as the “total daily carbohydrate rule”. The system defines a minimum, optimum and maximum daily carbohydrate content for a person, based on optimum weight vs. height requirements. The disclosure will henceforth refer to “required daily content” as the optimum daily carbohydrate intake needed by a person following the system and method of the present invention. The system provides a special portable dieting apparatus in the form of a food kit that comprises, in separate storage units, an asymmetric arrangement of a plurality of foods of known total carbohydrate content, correlated with the required daily content. The kit includes an entire range of foods, chosen from a large food list, which are preferably protected from spoiling by prevention means such as cooling packs. The only limitation on the type of food included is that the total weight of carbohydrates in the pyramid is less than a given number, preferably of the required daily content. The kit allows the user to have a varied diet, while making sure that the total carbohydrate daily intake is kept under a required limit.

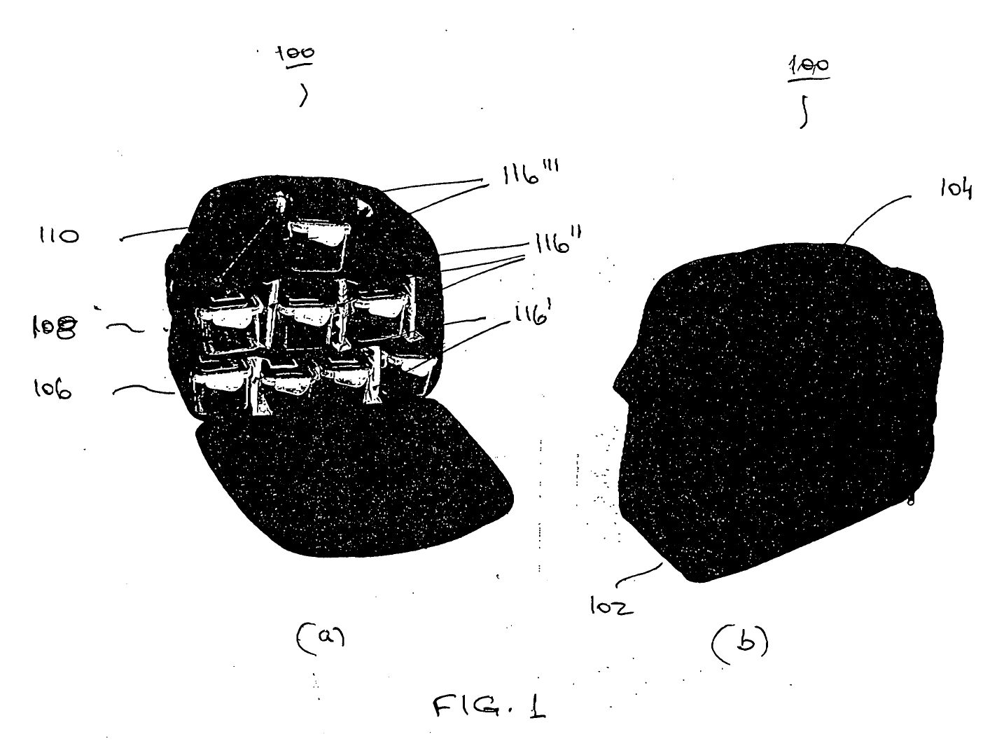

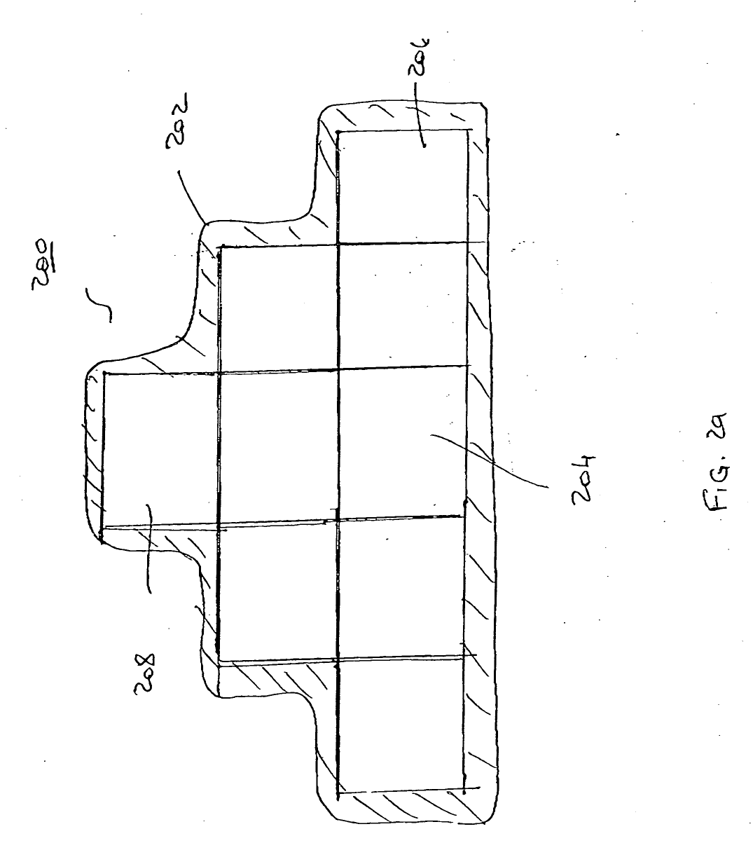

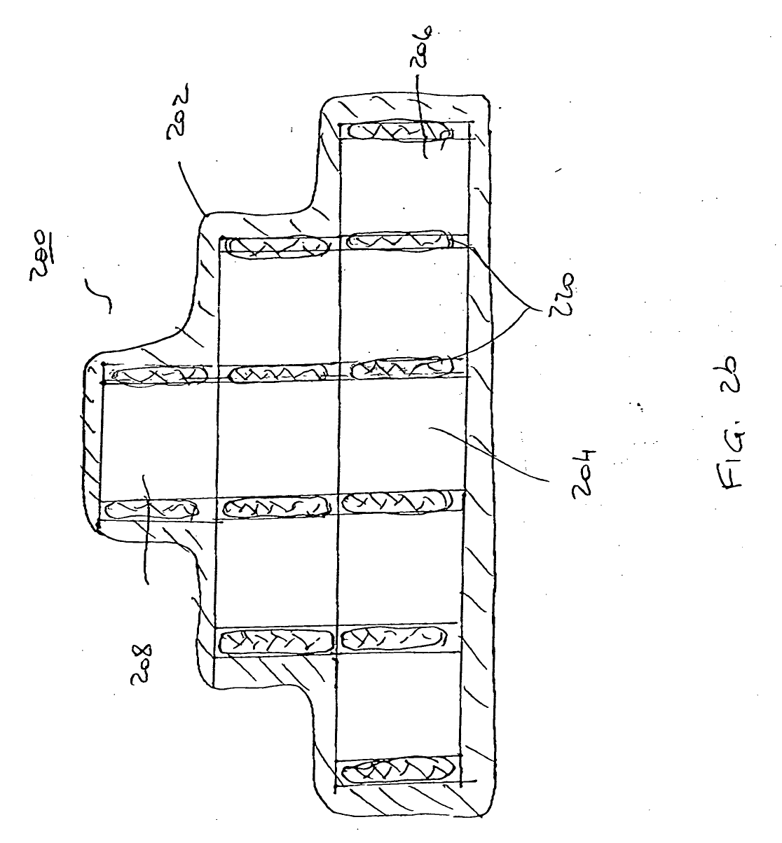

FIG. 1 is a photograph of a preferred embodiment of a portable dieting apparatus according to the present invention in the form of a kit 100: a) internal view; b) external view. Kit 100 has preferably a shape that allows stacking of a plurality of separate food container or bags, arranged in groups or “levels”. As used in this disclosure, a “container” implies any enclosure designed to separate one food from another. In this disclosure, “food” refers to both solid and liquid substances. Alternatively, as shown in FIG. 2, the containers may be just compartments in a one-piece enclosure, the enclosure having an asymmetric shape used for identifying the position of foods of a given carbohydrate content. Kit 100 can be made of any suitable material, either soft-sided or hard-sided. The kit preferably has an asymmetrical shape, most preferably resembling a truncated pyramid, with a substantially flat wide bottom side 102 and a substantially narrower top side 104. In this disclosure, “top” and “bottom” indicate spatial arrangement, and not necessarily a vertical structure. The largest group of containers (in a bottom level near the bottom side) preferably includes foods selected from the group of essentially carbohydrate free foods. The second largest group includes foods selected from the group of somewhat higher, limited carbohydrate content foods, the third largest group includes foods selected from the group of even higher carbohydrate content foods, etc. The top group (in a top level near the top side) typically includes one food selected from the group of highest carbohydrate content foods. In each group, each food is placed in a separate container. In the exemplary embodiment of FIG. 1, there are 8 such containers arranged in three groups, forming a pyramid of foods: a bottom group 106 that includes 4 containers of “no carbohydrate” foods that can be eaten freely, in any amount, a middle group 108 that includes 3 containers of “small carbohydrate amount” foods that have to be eaten in controlled, smaller amounts, and a top group 110 that includes one container of “carbohydrate-rich” food that has to be eaten in an extremely careful, controlled way. The number of groups and containers within each group is exemplary. It is understood, and within the scope of the present invention, that the kit may include a different number of stacked groups, and that each group may include a different number of containers than shown in FIG. 1. The constant guiding principle in any embodiment of the kit is that the group (level) with the largest number of containers includes foods selected from the group of lowest (or no) carbohydrate content, while the smallest group with the smallest number (preferably one) of containers includes a food selected from the group of highest carbohydrate content foods.

Returning to FIG. 1, in order to prevent spoilable foods (e.g. meats, milk, etc.) from being spoiled, the kit is preferably cooled by spoiling prevention means. These preferably include known cooling elements such as cooling packs that may be refrigerated, or any other known portable cooling devices. The groups are also preferably color-coded, for example by using different color containers (same color for all containers in a level), containers with different color covers (same cover color for all containers in the same level), or colored cooling packs disposed between or near the containers. In a preferred embodiment using colored cooling packs, bottom group 106 has disposed between its containers green cooling packs 116′, middle group 108 has disposed between its containers yellow cooling packs 116′″, and top group 110 has disposed around its single container red cooling packs 116″″. In this exemplary case, the green-yellow-red colors resemble traffic lights, providing the user with a visual reminder similar in meaning to those lights: thus, “green” means “go”, i.e. “eat freely low carbohydrate type foods”, “yellow” means “be careful” i.e. “eat fewer foods with larger carbohydrate content”, and “red” means “stop” i.e. “eat as little as possible from this type of high carbohydrate content food”.

FIG. 2a shows in cross section an embodiment of another portable dieting apparatus in the form of a kit 200 comprising an asymmetrical one-piece enclosure 202 having a plurality of compartments 204. The external shape need not follow the contour as shown, and may be similar to the one in FIG. 1. Clearly, the one-piece embodiment fulfills the same function of compartmentalizing foods of different carbohydrate content, carbohydrate-free foods being stored in a bottom level 206, a carbohydrate rich food being stored in a top level 208, and foods with increasing carbohydrate content being stored between the bottom level and the top level. Other shapes that provide a clear differentiation between various carbohydrate content foods are also within the scope of the present invention. The asymmetric shape itself may be enough to indicate which foods belong to the carbohydrate free group (i.e. the bottom level compartments), and which belong to the carbohydrate rich group (i.e. the top level compartment). Optionally, color means similar to those in FIG. 1 can be attached to each of the compartments or levels in FIG. 2a (not shown). Furthermore and preferably, spoiling prevention means, e.g. in the form of cooling packs, may be introduced inside one or more of the compartments of kit 200, or in specially designed spaces 220 provided between the compartments, as shown in FIG. 2b.

As mentioned, the kit allows the user to have a varied diet without worrying about calories, by making sure that the total carbohydrate daily intake is kept under a required limit. The content of foods in each container is chosen such that the total amount of food in the kit sums up to a required daily carbohydrate content. For simplicity and ease of use, this daily carbohydrate content is measured in “units” of 5 crams. The maximum daily carbohydrate content allowed for an obese or overweight person is preferably 16 units, i.e. a total of 80 grains of carbohydrates. The optimum carbohydrate allowed for the same person content is 12 units, i.e. 60 grams carbohydrates. The maximum and optimum number of units depends of course on the person's weight and height. Thus, obese or overweight children will obviously have smaller maximum and optimum allowed daily carbohydrate contents.

The amount of carbohydrates in each food is well known. In particular, various lists and other data sources that provide carbohydrate weight per total weight information are well known and readily accessible. Tables 1-3 below provide exemplary lists of no carbohydrate content foods (Table 1), moderate carbohydrate content foods (Table 2) and high carbohydrate content foods (Table 3). It is therefore quite simple to calculate the carbohydrate (and therefore the food) amount allowed at each level or group in the kit, so that the total carbohydrate content of the kit does not exceed 80 grams. Moreover, the kit would typically include less than the total carbohydrate amount allowed per diem, with the rest being for example allocated to home meals. Thus the kit may for example include foods stacked in groups as shown in FIG. 1, with a total carbohydrate content of 60 grams, while 20 grams of carbohydrates are allowed for home consumption that day.

TABLE 1
CARBOHYDRATE-FREE OR NEGLIGIBLE
CARBOHYDRATE CONTENT

| | |
|---|---|
| MEAT: | beef, lamb, turkey, veal, fowl, lamb, etc. |
| INTERNAL PARTS: | liver, kidneys, heart, tongue, brain, sausage |
| | meat, etc. |
| FISH: | tuna, salmon, sardines, cooked fish, salted fish, |
| | smoked fish, etc. |
| DAIRY AND FOWL | cheeses of all types, butter, eggs, salad oil, |
| PRODUCTS: | yoghurt, margarine, etc. |

TABLE 2
MEDIUM CARBOHYDRATE CONTENT FOODS

| ITEM | | QTY | | UNITS |
|---|---|---|---|---|
| AVOCADO | | 100 | GR | 1 |
| WATERMELON | | 100 | GR | 1 |
| NUTS | | 10 | GR | 0.5 |
| COCONUTS | | 50 | GR | 3 |
| PEARS | | 100 | GR | 2.5 |
| PINEAPPLES | | 100 | GR | 2 |
| GREENPEAS - COOKED | | 100 | GR | 2 |
| PERSIMON | | 25 | GR | 3 |
| PEACHES | | 100 | GR | 2 |
| ARTICHOKES | | 100 | GR | 1 |
| GRAPEFRUIT | | HALF | | 2 |
| PEANUTS - ROASTED | | 30 | GR | 1 |
| BAMIA - COOKED | | 100 | GR | 1 |
| GUAVAS | | 50 | GR | 2 |
| CARROTS | | 80 | GR | 1 |
| SUNFLOWER SEEDS | | 25 | GR | 1 |
| CHERRIES | | 100 | GR | 3 |
| MILK | FULL CREAM | 100 | GR | 1 |
| | SKIM | 100 | GR | 1 |

| | | | |
|---|---|---|---|
| PEANUT BUTTER | 25 | GR | 1 |
| EGGPLANT | 100 | GR | 1 |
| TEHINA | 50 | GR | 0.5 |
| LEMONS | 100 | GR | 1.5 |
| BREAD - LOW CALORIE | 25 | GR | 1.5 |
| PINEAPPLE JUICE | 100 | | 2.5 |
| TOMATO JUICE | 100 | | 1 |
| ORANGE JUICE | 100 | | 2 |
| MELON | 170 | | 2 |
| MANGOES | 100 | | 2 |
| MANDARINES | 100 | | 2 |
| SOUPS | 200 | GR | 2-0 |
| APRICOTS | 50 | GR | 1 |
| BEETROOTS | 50 | GR | 1 |
| LENTILS | 25 | GR | 3 |
| CHESTNUTS | 50 | GR | 4 |
| RASPBERRIES | 100 | GR | 2 |
| FALAFEL | 50 | GR | 2 |
| CACTUS FRUIT | 50 | GR | 1 |
| RAISINS | 25 | GR | 3 |
| CLEMENTINES | 100 | GR | 2 |
| SOYA FLOUR | 25 | GR | 1 |
| POMEGRANATES | 50 | GR | 2 |
| PLUMS | 100 | GR | 2 |
| ALMONDS | 30 | GR | 1 |
| FIGS | 100 | GR | 3 |
| ORANGES | 200 | GR | 4 |
| STRAWBERRIES | 100 | GR | 2.5 |

TABLE 3
HIGH CARBOHYDRATE CONTENT FOODS

| ITEM | | QTY | | UNITS |
|---|---|---|---|---|
| PEARS IN SYRUP | | 100 | GR | 5 |
| RICE - COOKED | | 100 | GR | 5 |
| PINEAPPLE IN SYRUP | | 100 | GR | 5 |
| PEACHES IN SYRUP | | 100 | GR | 5 |
| PEACHES DRIED | | 50 | GR | 7 |
| NOODLES - COOKED | | 175 | GR | 9 |
| BITTER LEMON | | 100 | GR | 6 |
| BISCUITS | | 2 | | 3 |
| BEER | | 1 | PINT GLASS | 2 |
| BANANAS | | 1 | | 5 |
| BRANDY | | 25 | GR | 4 |
| GIN | | 25 | GR | 4 |
| GINGER ALE | | 25 | GR | 6 |
| JELLY | | 100 | GR | 4 |
| ICECREAM | | 50 | GR | 2 |
| HONEY | | 25 | GR | 5 |
| WHISKEY | | 25 | GR | 4 |
| VERMOUTH - SWEET | | 50 | GR | 5 |
| VERMOUTH - DRY | | 50 | GR | 3 |
| SEMOLINA POWDER | | 25 | GR | 4 |
| SEMOLINA PUDDING | | 100 | GR | 4 |
| DOUGHNUTS | | 50 | GR | 6 |
| CYDER | | 1 | GLASS | 3.5 |
| SYRUP | | 15 | GR | 2.5 |
| SPAGETTI - COOKED | | 170 | GR | 10 |
| CAKES | DRY | 50 | GR | 7 |
| | WITH CREAM | 50 | GR | 7 |
| | WITH ICING | 50 | GR | 8 |
| | WITH FRUIT | 50 | GR | 7 |
| | APPLE PIE | 50 | GR | 3.5 |

| GRAPES | | 100 | GR | 3 |
|---|---|---|---|---|
| PUDDING | POWDER | 100 | GR | 2 |
| | WITH EGG | 100 | GR | 2 |
| | WITH MILK | 100 | GR | 5 |

| BEANS - DRIED | | 50 | GR | 5 |
|---|---|---|---|---|
| RASPBERRY SYRUP | | 100 | GR | 5 |
| PITA BREAD | | 25 | GR | 3 |
| PANCAKES | | 50 | GR | 4 |
| FRUIT IN SYRUP | | 100 | GR | 5 |
| HUMUS - CHICKPEAS | | 50 | GR | 6 |
| MILK - CONCENTRATED | | 100 | GR | 2 |
| HALVAH | | 50 | GR | 5 |
| CHALLAH | | 25 | GR | 3 |
| TAPIOCA | UNCOOKED | 100 | GR | 4 |
| | PUDDING | 100 | GR | 5 |

| YOGURT - FRUIT-CHOCOLATE | | 100 | GR | 3 |
|---|---|---|---|---|
| WINE | DRY | 50 | GR | 3 |
| | SWEET | 50 | GR | 4 |
| BREAD | MEDIUM SLICED | | | |
| | DARK | 25 | GR | 3 |
| | WHITE | 25 | GR | 3 |

| BUNS | | 50 | GR | 6 |
|---|---|---|---|---|
| LEMONADE | | 225 | GR | 5 |
| LIQUER | | 25 | GR | 5 |
| TONIC WATER | | 170 | GR | 2.5 |
| GRAPEFRUIT JUICE - SWEET | | 100 | GR | 3 |
| ORANGE JUICE - SWEET | | 100 | GR | 3 |
| MANDARINES IN SYRUP | | 100 | GR | 4 |
| MATZOS | | 25 | GR | 3.5 |
| MACARONI | COOKED | 170 | GR | 10 |
| | WITH CHEESE | 100 | GR | 5 |

| MARMALADE | | 15 | GR | 4 |
|---|---|---|---|---|
| APRICOTS | IN SYRUP | 100 | GR | 5 |
| | DRIED | 50 | GR | 6 |
| SUGAR | BROWN | 15 | GR | 3 |
| | WHITE | 15 | GR | 3 |

| TOFFEE SWEETS | | 25 | GR | 4 |
|---|---|---|---|---|
| CORNFLAKES | | 25 | GR | 4 |
| BREAD TOASTS | | 25 | GR | 3 |
| QUAKER OATS | UNCOOKED | 25 | GR | 3 |
| | COOKED | 100 | GR | 3 |

| COCA COLA | | 100 | GR | 3 |
|---|---|---|---|---|
| CORNFLOUR | | 25 | GR | 5 |
| FLOUR | BARLEY | 25 | GR | 4 |
| | OATS | 25 | GR | 4 |
| | CORN | 25 | GR | 4 |
| | POTATO | 25 | GR | 4 |
| | RICE | 25 | GR | 4 |
| | WHEAT | 25 | GR | 4 |
| COCOA | | 25 | GR | 2.5 |
| JAMS | | 15 | GR | 2 |
| MILK CHOCOLATE | | 50 | GR | 5 |
| BITTER CHOCOLATE | | 50 | GR | 5 |
| PLUMS | DRIED | 50 | GR | 7 |
| | IN SYRUP | 100 | GR | 5 |

| YEAST | | 100 | GR | 7.5 |
|---|---|---|---|---|
| CHERRIES | | 50 | GR | 4 |
| FIGS IN SYRUP | | 100 | GR | 8 |
| FIGS DRIED | | 25 | GR | 3 |
| STRAWBERRIES IN SYRUP | | 100 | GR | 5 |
| CORN ON COB - COOKED | | 100 | GR | 4 |
| DATES - DRIED | | 25 | GR | 4 |
| POTATOES | COOKED | 100 | GR | 4 |
| | CHIPS | 25 | GR | 3 |

| | | | |
|---|---|---|---|
| SWEET POTATOES - BAKED | 100 | GR | 7 |
| APPLES - DRIED | 25 | GR | 3 |

Note:
carbohydrate value marked in units
* 1 unit = 5 grams carbohydrate
** alcoholic drinks are carbohydrate free but high-caloried, which raises the number of units.

In use, a user would typically prepare a kit each day by choosing foods to be stored in each container of each level, from a list of items with known carbohydrate content. For this purpose, one may use any list of foods that provides the carbohydrate content, for example Tables 1-3 herein. One may form example choose to fill the four containers of the “no carbohydrate” bottom group 106 in FIG. 1 with items from Table 1 above such as beef, chicken, veal, lamb, turkey, etc., each of which has little or no carbohydrates, fish, cheeses, vegetables, and other known “no carbohydrate” foods. Since these foods do not have any (or very little) carbohydrate content, they may be eaten in basically unlimited amounts. Practically, the amounts will be limited by the volume of the bottom group containers, but the user may eat more of these foods at home or in a restaurant, without affecting the diet regimen. Similarly, the 3 middle group 108 containers in FIG. 1 may be filled with 100 grams of avocado (equivalent to one carbohydrate unit or simply “one unit”—see Table 2), 30 grams of roasted peanuts (one unit), and 200 grams of oranges (4 units), for a total of 6 carbohydrate units. According to the “total daily carbohydrate rule”, there are 6 more units allowed to reach the optimum daily carbohydrate intake, and 10 more units to reach the maximum allowed daily intake. The top group (110) container may thus include foods from Table 3 such as a bun (50 grams weight, 5 carbohydrate units) plus 15 grams of jam (2 units), 100 grams of fruit in syrup (e.g. peaches)=5 units, etc. As mentioned, the kit may contain less than the optimum daily intake, with the rest eaten at home or in a restaurant. However, by stacking the kit each day with foods arranged in a pyramid according to their carbohydrate (per unit weight) content, one makes sure that the diet regimen is followed optimally.

In summary, in contrast with existing and known diet system and methods, the present invention provides a diet regimen based on a defined, -limited carbohydrate daily intake. The system organizes foods according to carbohydrate content in a kit. The carbohydrate content is preferably defined by units, each food having a known number of carbohydrate units per food weight unit. The user can then freely eat the foods in the kit throughout the day. This diet does not count calories - in fact, the number of calories in the foods eaten is not important. The principle behind the system and method disclosed herein is the controlled daily intake of carbohydrates spread among a wide variety of foods, the organizing of these foods in a portable kit that has means to distinguish between the various carbohydrate content groups, and means to prevent the spoiling of these foods.

While the invention has been described with respect to a limited number of embodiments, it will be appreciated that many variations, modifications and other applications of the invention may be made.

1. A system for reducing human body weight to a permanent optimal weight level, comprising:
a. a portable kit that includes a plurality of foods with a known total carbohydrate content; and
b. spoiling prevention means for keeping said foods from spoiling.

2. The system of claim 1, wherein said plurality of foods is organized in groups of foods characterized by a substantially similar carbohydrate content, each of said foods contained in a separate container.

3. The system of claim 2, wherein said containers of each said group of foods characterized by a substantially similar carbohydrate content are stacked in levels, from a bottom level that includes a maximal number of containers with foods characterized by a substantially nil carbohydrate content, to a top level that includes a minimal number of containers with foods characterized by a high carbohydrate content.

4. The system of claim 3, wherein said number of levels includes three levels, said bottom level, a middle level and said top level, and wherein said bottom level includes four said containers, said middle level includes three said containers, and said top level includes one said container.

5. The system of claim 4, wherein said spoiling prevention means include cooling packs.

6. The system of claim 5, wherein said cooling packs are arranged in accordance with said levels to provide optimal cooling to each said container.

7. The system of claim 6, wherein each said level is color-coded.

8. The system of claim 7, wherein said color-coding is provided by said cooling packs being colored with a different color for each said level.

9. The system of claim 7, wherein said color coding include red for said top level, yellow for said middle level and green for said bottom level.

10. A method for reducing body weight in humans comprising the steps of:
a. determining an optimal daily carbohydrate intake;
b. organizing foods in a portable device according to said optimal carbohydrate intake; and
c. freely consuming said food throughout the day, whereby the method provides permanent weight loss.

11. The method of claim 10, further comprising the step of cooling said foods to prevent spoiling.

12. The method of claim 11, wherein said step of organizing foods in a portable device includes providing a kit configured to carry food containers arranged in levels, each said level defined by foods with a substantially similar carbohydrate content.

13. The method of claim 12, wherein said step of cooling includes positioning cooling packs between said containers in each said level.

14. The method of claim 13, wherein said kit configuration includes a bottom level with a maximal number of substantially carbohydrate free foods, and additional stacked levels of progressively fewer said containers stacked above said bottom level, said stacking ending with a top level having a minimal number of said containers filled with foods of a maximal carbohydrate content.

15. The method of claim 14, wherein said additional stacked levels include a middle level inserted between said bottom and said top levels.

16. The method of claim 15, wherein said positioning of said cooling packs between said containers in said three levels includes positioning color coded packs, each said level including packs of a similar color.

17. The method of claim 16, wherein said positioning of color coding packs includes positioning green packs in said bottom level, yellow packs in said middle level and red packs in said top level.

18. A portable dieting apparatus comprising:
a. an asymmetrically shaped kit that includes a plurality of food storage units; and
b. a plurality of foods having a known total carbohydrate content stored in said storage units according to a carbohydrate content order.

19. The apparatus of claim 18, wherein said storage units are separate containers.

20. The apparatus of claim 18, wherein said storage units include separate compartments in a one-piece enclosure.

21. The apparatus of claim 19, further comprising spoiling prevention means to keep said foods from spoiling.

22. The apparatus of claim 21, further comprising color-coding means for marking said carbohydrate content order.

23. The apparatus of claim 18, wherein said asymmetric shape includes a substantially pyramid shape with a wide bottom and a narrow top, and wherein said plurality of foods stored in said storage units according to a carbohydrate content order include carbohydrate-free foods stored in storage units near said bottom and at least one carbohydrate rich food stored in at least one storage unit near said top.
